(19) United States
(12) Patent Application Publication
(10) Pub. No.: US 2026/0183011 A1
(43) Pub. Date: Jul. 2, 2026

(54) LINE-DRIVEN MOTION MODULE AND MINIMALLY INVASIVE SURGICAL FORCEPS

(71) Applicant: JINGQIN ZHIZAO (SUZHOU) MEDICAL TECHNOLOGY CO., LTD., Suzhou, Jiangsu (CN)

(72) Inventors: Yichu WANG, Suzhou (CN); Xiaozhen LI, Suzhou (CN); Jingyao DAI, Suzhou (CN); Xinliangyi XU, Suzhou (CN)

(73) Assignee: JINGQIN ZHIZAO (SUZHOU) MEDICAL TECHNOLOGY CO., LTD., Suzhou, Jiangsu (CN)

(21) Appl. No.: 18/836,412

(22) PCT Filed: May 4, 2023
(86) PCT No.: PCT/CN2023/092074

§ 371 Date: Aug. 7, 2024

(30) Foreign Application Priority Data
Aug. 31, 2022 (CN) 202211062457.6

(51) Int. Cl. A61B 17/29; A61B 17/00

(52) U.S. Cl. CPC A61B 17/29; A61B 2017/00367; A61B 2017/00477; A61B 2017/2905; A61B 2017/2908

(57) ABSTRACT

A line-driven motion module includes a joint structure, a positioning structure, and two connecting lines. The joint structure has interconnected joint units. The positioning structure is arranged between two adjacent joint units and includes first and second positioning members that are movably connected to each other and move towards or away from each other. The connecting lines are arranged in the joint units and the positioning structure in a penetrating manner. Both ends of any connecting line are in positioning connection to the first and second positioning members, respectively, to limit the length of the connecting line connected into the positioning structure to be constant. A minimally-invasive surgical forceps device uses the line-driven motion module. The line drive motion module can adjust the motion trajectory of the joint structure, widens the motion range of the joint structure, and enhances the flexible operation performance of a minimally invasive device.

### CROSS-REFERENCE TO RELATED APPLICATIONS

The present application claims priority to Chinese Patent Application No. 202211062457.6, filed to the China Patent Office on Aug. 31, 2022 and entitled “LINE-DRIVEN MOTION MODULE AND MINIMALLY INVASIVE SURGICAL FORCEPS”, the entire content of which is incorporated herein by reference.

### TECHNICAL FIELD

The present application relates to the technical field of medical instruments, in particular to a line-driven motion module and minimally invasive surgical forceps.

### BACKGROUND

A joint structure is widely applied in the fields of machinery and medical care due to its good strength and capability of flexible adaptability to space motion. At present, in medical instruments, for example, in minimally invasive surgery, a doctor enables a minimally invasive device to perform expected actions according to control actions and instructions through a corresponding operating mechanism, so as to achieve the purpose of medical treatment and diagnosis for a human body.

In the prior art, the minimally invasive device is usually composed of a joint structure and a plurality of connecting lines, the connecting lines are the joint structure are connected inside, one ends of the plurality of connecting lines are connected with an external driving device, and the other ends of the plurality of connecting lines are connected with an external operating end. When a joint structure of a minimally invasive machine needs to be adjusted to bend, the connecting lines are withdrawn through the external driving device, adjacent joints where a line withdrawing end is located approach to one another, and an included angle between the adjacent joints is reduced; the connecting lines are pushed and extended through the external driving device, adjacent joints where a line pushing end is located move away from one another, and an included angle between the adjacent joints is increased; and accordingly, bending and folding actions of the joints are completed, and an expected operating process is achieved.

However, if the above structure is adopted, when the driving device controls the joint structure to perform actions, a motion trajectory of a distal node of the joint structure remains on a spherical surface with a fixed point as a center of sphere, and the distal node of the joint structure can move only on this spherical surface, which limits the motion trajectory of the joint structure; when the doctor performs operations such as clamping and suturing in a length extending direction from a proximal end to a distal end, the distal node of the joint structure needs to be displaced in this length direction, the above structure is hardly adapted to the displacement in this length direction, a motion range is insufficient, and a problem of limiting flexible operation performance of the minimally invasive device exists.

### SUMMARY OF THE INVENTION

A technical problem to be solved by the present application is a defect that in the prior art, a motion trajectory of the joint structure is limited, a motion range is insufficient, and flexible operations of the minimally invasive device is limited.

In a first aspect, the present application provides a line-driven motion module, including:
a joint structure, wherein one end of the joint structure is configured to be connected with an external driving mechanism, the other end of the joint structure is configured to be connected with an external operating mechanism, and the joint structure includes a plurality of interconnected joint units;
a positioning structure, wherein the positioning structure is arranged between two adjacent joint units, the positioning structure includes a first positioning member and a second positioning member, and the first positioning member and the second positioning member are movably connected to each other and can move towards or away from each other in a first direction;
at least two connecting lines, wherein the connecting lines are arranged in all the joint units and the positioning structure in a penetrating manner, each of the connecting lines is arranged to pass through two ends of the positioning structure, and the first positioning member and the second positioning member are configured to limit and restrain a length of each of the connecting lines inside the positioning structure;
wherein, when the first positioning member and the second positioning member move towards each other in the first direction, a length of each of the connecting lines is limited and received in a line receiving cavity co-enclosed by the first positioning member and the second positioning member; and when the first positioning member and the second positioning member move away from each other in the first direction, the length of each of the connecting lines is straightened in the line receiving cavity.

Optionally, in the line-driven motion module, the positioning structure further includes at least two positioning sleeves, the positioning sleeves are arranged in correspondence with the connecting lines and configured to limit and receive the connecting lines, the positioning sleeves are arranged inside the line receiving cavity, and two ends of each positioning sleeve are connected with a corresponding connecting line at a first line connecting port of the first positioning member and at a second line connecting port of the second positioning member, respectively.

Optionally, in the line-driven motion module, the positioning sleeves are flexible tubes;
when the first positioning member and the second positioning member move towards each other in the first direction, the flexible tubes are folded or coiled or curled synchronously with the connecting lines inside the line receiving cavity; and
when the first positioning member and the second positioning member move away from each other in the first direction, the flexible tubes are unfolded or stretched synchronously with the connecting lines.

Optionally, the line-driven motion module further comprises a first adapter structure and a second adapter structure which are spaced apart from each other, the first adapter structure and the second adapter structure are arranged at two ends of the joint structure, the first adapter structure is configured to be connected with the external driving mechanism, and the second adapter structure is configured to be connected with the operating mechanism; and
the line-driven motion module has bent states in which the first adapter structure draws the connection lines under an external force to enable the second adapter structure and the joint structure to bend in a linked manner synchronously with respect to the first adapter structure, and an initial state in which the first adapter structure, the positioning structure and the second adapter structure are arranged coaxially; and the line-driven motion module is arranged to switch between the bent states and the initial state.

Optionally, in the line-driven motion module, the first positioning member is provided with a first connecting portion, and the second positioning member is provided with a second connecting portion; and
the first connecting portion and the second connecting portion are mated screw thread structures, and when the first connecting portion and the second connecting portion are in threaded connection, the first positioning member and the second positioning member are relatively movable towards each other or away from each other in the first direction, the first direction being an axial direction of the screw thread structures.

Optionally, in the line-driven motion module, the first positioning member and the second positioning member are tubular structures which are nested together, the first connecting portion is formed on an inner wall surface of the first positioning member; the second connecting portion is formed on an outer wall surface of the second positioning member; and the line receiving cavity is a cavity within the tubular structures.

Optionally, in the line-driven motion module, each connecting line is arranged in a corresponding first line connecting port of the first positioning member and a corresponding second line connecting port of the second positioning member in a penetrating manner; and when the first positioning member is screwed to a first rotation position relative to the second positioning member, each first line connecting port and the corresponding second line connecting port are on a line in the same direction as the first direction, and the line-driven motion module is in a first bent state in which the first adapter structure draws the connection lines under an external force to enable the second adapter structure and the joint structure to bend in a linked manner to move in an S shape synchronously with respect to the first adapter structure.

Optionally, in the line-driven motion module, in the first bent state, a first end surface, remote from a side thereof connected to the joint structure, of the first adapter structure, is arranged parallel to a second end surface, remote from a side thereof connected to the joint structure, of the second adapter structure.

Optionally, in the line-driven motion module, each connecting line is arranged in a corresponding first line connecting port of the first positioning member and a corresponding second line connecting port of the second positioning member in a penetrating manner; and when the first positioning member is screwed to a second rotation position relative to the second positioning member, a first plane containing one first line connecting port and the first direction is oriented at an angle of 90 degrees to a second plane containing the corresponding second line connecting port and the first direction, and the line-driven motion module is in a second bent state in which the first adapter structure draws the connection lines under an external force to enable the second adapter structure and the joint structure to bend in a linked manner to move in a noncoplanar S shape synchronously with respect to the first adapter structure.

Optionally, in the line-driven motion module, each connecting line is arranged in a corresponding first line connecting port of the first positioning member and a corresponding second line connecting port of the second positioning member in a penetrating manner; and when the first positioning member is screwed to a third rotation position relative to the second positioning member, a first plane containing one first line connecting port and the first direction is oriented at an angle of 180 degrees to a second plane containing the corresponding second line connecting port and the first direction; and
the line-driven motion module is in a third bent state in which the first adapter structure draws the connection lines under an external force to enable the second adapter structure and the joint structure to bend in a linked manner to move in a C shape synchronously with respect to the first adapter structure.

Optionally, in the line-driven motion module, the first positioning member is provided with a third connecting portion, and the second positioning member is provided with a fourth connecting portion; and
the third connecting portion and the fourth connecting portion are mated slidable structures, when the third connecting portion and the fourth connecting portion slide relative to each other, the first positioning member and the second positioning member are relatively movable towards each other or away from each other in the first direction, and the first direction is a sliding direction of the slidable structures.

Optionally, the line-driven motion module further includes a swiveling state in which the first adapter structure is rotated under an external force to draw the connecting lines and enable the second adapter structure and the joint structure to twist synchronously with the first adapter structure.

Optionally, in the line-driven motion module, each joint unit includes:
a rotating member provided with limiting portions in a circumferential direction; and
a limiting base fixedly connected to the rotating member, wherein the limiting base is internally provided with at least one receiving cavity for being movably connected to the rotating member of an adjacent joint unit, the receiving cavity is further internally provided with limiting members, and the limiting members cooperate with the limiting portions of the adjacent joint unit to limit axial rotation of the rotating member of the adjacent joint unit.

Optionally, in the line-driven motion module, the rotating member is a sphere, an inner wall surface of the receiving cavity is fitted with an outer wall surface of the rotating member of the adjacent joint unit;
two limiting portions are provided, and the two limiting portions cooperate to form the sphere; and
the two limiting portions are spaced apart to form a fitting space for limiting the limiting members, and the limiting members are movably connected into the fitting space.

Optionally, in the line-driven motion module, the first positioning member includes a first connecting hole extending in the same direction as an extending direction of the first positioning member;
the second positioning member includes a second connecting hole extending in the same direction as an extending direction of the second positioning member;
each of the adapter structures is provided with a third connecting hole extending in the same direction as an extending direction of the adapter structure; and
each of the joint units is provided with a core hole, and all the core holes, all the second connecting holes and all the first connecting holes together form an intervention channel configured for an externally-connected pipe to penetrate therethrough.

Optionally, in the line-driven motion module, the first positioning member further includes a first joining portion arranged on a side, close to a proximal end, of the first positioning member, and the first joining portion is movably connected to a distal end of a first joint structure;
the second positioning member further includes a second joining portion arranged on a side, away from the proximal end, of the second positioning member, and the second joining portion is movably connected to a proximal end of the joint structure; and
the first joining portion and the second joining portion are respectively identical in structure to the rotating member or identical in structure to the limiting base.

Optionally, in the line-driven motion module, the first adapter structure includes a third joining portion movably connected with a joint unit;
the second adapter structure includes a fourth joining portion movably connected with a joint unit; and
the third joining portion and the fourth joining portion are respectively identical in structure to the rotating member or identical in structure to the limiting base.

In a second aspect, the present application provides minimally invasive surgical forceps, including the line-driven motion module in the first aspect.

The technical solutions provided by the present application have the following advantages.
1. The line-driven motion module provided by the present application includes the joint structure, the positioning structure and the two connecting lines, one end of the joint structure is configured to be connected with an external driving mechanism, the other end of the joint structure is configured to be connected with an external operating mechanism, and the joint structure includes a plurality of interconnected joint units; the positioning structure is arranged between two adjacent joint units, the positioning structure includes the first positioning member and the second positioning member, and the first positioning member and the second positioning member are movably connected to each other and can move towards or away from each other in the first direction; the connecting lines are arranged in all the joint units and the positioning structure in a penetrating manner, each of the connecting lines is arranged to pass through two ends of the positioning structure, and the first positioning member and the second positioning member are configured to limit and restrain a length of each of the connecting lines inside the positioning structure; wherein, when the first positioning member and the second positioning member move towards each other in the first direction, a length of each of the connecting lines is limited and received in the line receiving cavity co-enclosed by the first positioning member and the second positioning member; and when the first positioning member and the second positioning member move away from each other in the first direction, the length of each of the connecting lines is straightened in the line receiving cavity.

In the line-driven motion module of this structure, the first positioning member and the second positioning member are movably connected to each other so as to move towards or away from each other in the first direction, thus a joint structure connected to the first positioning member is correspondingly displaced to move towards or away from a joint structure connected to the second positioning member in the first direction, a motion position of the joint structure is adjusted through motion of the first positioning member and the second positioning member, and a purpose of adjusting a motion trajectory of the joint structure in the first direction through the positioning structure is achieved; the connecting lines inside the positioning structure are limited and restrained through positioning cooperation of the first positioning member and the second positioning member, and specifically, when the first positioning member and the second positioning member move towards each other in the first direction, the connecting lines are limited to be inside the line receiving cavity, and the joint structure performs withdrawal folding motion; when the first positioning member and the second positioning member move away from each other in the first direction, the connecting lines are released out of the line receiving cavity, so that the connecting lines are extended and straightened, and the joint structure performs extending and expanding motion; and the first positioning member and the second positioning member limit and restrain the connecting lines to be in a length of a distance between two ends of the positioning structure, a length of each of the connecting lines inside the positioning structure remains constant and does not change even though the first positioning member and the second positioning member slide in the first direction, change motion of each of the connecting lines inside the positioning structure is fitted with a relative motion of the first positioning member and the second positioning member and a position of the motion trajectory of each of the joint structures at two ends of the positioning structure, the motion trajectory of the joint structure is optimized, the motion range of the joint structure is widened, and thus a purpose of enhancing the flexible operation performance of the minimally invasive device is achieved.
2. In the line-driven motion module provided by the present application, the positioning structure further includes two positioning sleeves, the positioning sleeves are arranged in correspondence with the connecting lines and configured to limit and receive the connecting lines, the positioning sleeves are arranged inside the line receiving cavity, and two ends of each positioning sleeve are connected with a corresponding connecting line at a first line connecting port of the first positioning member and at a second line connecting port of the second positioning member, respectively; the positioning sleeves are flexible tubes; when the first positioning member and the second positioning member move towards each other in the first direction, the flexible tubes are folded or coiled or curled synchronously with the connecting lines inside the line receiving cavity; and when the first positioning member and the second positioning member move away from each other in the first direction, the flexible tubes are unfolded or stretched synchronously with the connecting lines.

In the line-driven motion module of this structure, the first positioning member, the second positioning member and the positioning sleeves cooperate jointly to limit the motion of the connecting lines, two ends of the positioning sleeves are fixedly connected with the first positioning member and the second positioning member respectively, the connecting lines are arranged inside the positioning sleeves in a penetrating manner, axial displacement of the connecting lines is limited through the positioning sleeves, and the positioning sleeves adopt the flexible members, so when the first positioning member and the second positioning member move towards each other in the first direction, the first positioning member and the second positioning member drive two ends of each positioning sleeve to move towards each other relative to a middle of the positioning sleeve, and at the same time, the positioning sleeves limit and squeeze the connecting lines therein, so that the connecting lines are folded or coiled or curled synchronously with the positioning sleeves inside the line receiving cavity; and when the first positioning member and the second positioning member move away from each other in the first direction, the first positioning member and the second positioning member drive the two ends of each positioning sleeve to move away from each other relative to the middle of the positioning sleeve, at the same time, the two ends of the positioning sleeves limit and stretch the connecting lines therein, so that the connecting lines are unfolded or stretched synchronously with the positioning sleeves, displacement of the connecting lines matches a distance between the first positioning member and the second positioning member, and the motion range of the joint structure can be widened.
3. The line-driven motion module provided by the present application further includes the first adapter structure and the second adapter structure which are spaced apart from each other, the first adapter structure and the second adapter structure are arranged at two ends of the joint structure, the first adapter structure is configured to be connected with the external driving mechanism, and the second adapter structure is configured to be connected with the operating mechanism; all the connecting lines have the same length; the line-driven motion module has bent states in which the first adapter structure draws the connection lines under an external force to enable the second adapter structure and the joint structure to bend in a linked manner synchronously with respect to the first adapter structure, and an initial state in which the first adapter structure, the positioning structure and the second adapter structure are arranged coaxially; and the line-driven motion module is arranged to switch between the bent states and the initial state.

In the line-driven motion module of this structure, the external driving mechanism drives and adjusts a motion position of the first adapter structure, and the second adapter structure is transmitted through cooperation of the connecting lines and the joint structures; when the line-driven motion module is switched between the bent state and the initial state, the first adapter structure and the second adapter structure perform corresponding motion around the positioning structure, the connecting lines are arranged in the same length, so that the second adapter structure and the first adapter structure are linked synchronously, and the second adapter structure achieves expected motion actions and positions; through an integral module design, stability of the connecting lines driving the joint units is improved, the tight connection is beneficial for improving connection strength of the joint structure, and integral motion performance of the line-driven motion module is balanced.

### BRIEF DESCRIPTION OF THE DRAWINGS

In order to more clearly describe specific implementations of the present application or technical solutions in the prior art, the accompanying drawings needed by the description in the specific implementations or in the prior art will be briefly introduced below. Apparently, the accompanying drawings in the following description are some implementations of the present application. Those ordinarily skilled in the art can also obtain other accompanying drawings according to these accompanying drawings without making creative work.

FIG. 1 is a schematic structural diagram of a line-driven motion module provided by an embodiment of the present application.

FIG. 2 is a schematic diagram of connection of a positioning structure in a line-driven motion module provided by an embodiment of the present application.

FIG. 3 is a schematic three-dimensional structural diagram of a first positioning member in a line-driven motion module provided by an embodiment of the present application.

FIG. 4 is a schematic structural side view of a first positioning member in a line-driven motion module provided by an embodiment of the present application.

FIG. 5 is a schematic structural front view of a first positioning member in a line-driven motion module provided by an embodiment of the present application.

FIG. 6 is a schematic three-dimensional structural diagram of a second positioning member in a line-driven motion module provided by an embodiment of the present application.

FIG. 7 is a schematic structural front view of a second positioning member in a line-driven motion module provided by an embodiment of the present application.

FIG. 8 is a schematic structural side view of a second positioning member in a line-driven motion module provided by an embodiment of the present application.

FIG. 9 is a schematic structural diagram of a line-driven motion module in a first bent state provided by an embodiment of the present application.

FIG. 10 is a schematic structural diagram of a line-driven motion module in a second bent state provided by an embodiment of the present application.

FIG. 11 is a schematic structural diagram of a joint unit in a line-driven motion module provided by an embodiment of the present application.

FIG. 12 is a schematic three-dimensional diagram of a joint unit in a line-driven motion module provided by an embodiment of the present application.

FIG. 13 is a schematic structural diagram of a first adapter structure in a line-driven motion module provided by an embodiment of the present application.

FIG. 14 is a schematic structural diagram when a first adapter structure in a line-driven motion module is located on a distal end side provided by an embodiment of the present application.

### DESCRIPTIONS OF REFERENCE NUMERALS

1—joint unit; 11—rotating member; 111—limiting portion; 12—limiting base; 121—receiving cavity; 122—core hole; 123—mounting hole; 13—limiting member;
2—first positioning member; 21—first line connecting port; 22—first connecting portion; 23—first connecting hole; 24—first joining portion;
3—second positioning member; 31—second line connecting port; 32—second connecting portion; 33—second connecting hole; 34—second joining portion;
4—connecting line; 5—positioning sleeve;
6—first adapter structure; 61—third joining portion; 62—first adapter body; 621—third connecting hole; 622—fitting hole; 623—first end surface;
7—second adapter structure; 71—fourth joining portion; 72—second adapter body; 721—second end surface.

### DETAILED DESCRIPTION

The technical solutions of the present application are clearly and completely described in the following with reference to the accompanying drawings. Apparently, the described embodiments are merely some rather than all of the embodiments of the present application. All other embodiments obtained by those ordinarily skilled in the art based on the embodiments of the present application without making creative efforts fall within the protection scope of the present application.

In the description of the present application, it needs to be noted that directions or position relations indicated by terms such as “center”, “upper”, “lower”, “left”, “right”, “vertical”, “horizontal”, “inner” and “outer” are directions or position relations as shown in the accompanying drawings and are only intended to conveniently describe the present application and simplify the description but not to indicate or imply that an apparatus or element referred to necessarily has a specific direction or is constructed or operated in a specific direction, so as not to be understood as a limitation on the present application. Besides, terms such as “first”, “second” and “third” are only used for the description instead of being understood as indicating or implying a relative significance.

In the description of the present application, it needs to be noted that unless otherwise specified and defined clearly, terms such as “connection”, “connected”, “connecting” are to be understood in a broad sense, for example, it may be a fixed connection, or detachable connection, or an integrated connection; it may be a mechanical connection or an electrical connection; and it may be a direct connection, or an indirect connection through an intermediate medium, or internal communication between two elements. Specific meanings of the above terms in the present application may be understood by those ordinarily skilled in the art according to specific conditions.

In the following description, when in use, a side of an apparatus close to an operator is a proximal end, and an end away from the operator is a distal end.

Besides, technical features involved in different implementations of the present application described below may be combined mutually without a conflict between one another.

### Embodiment 1

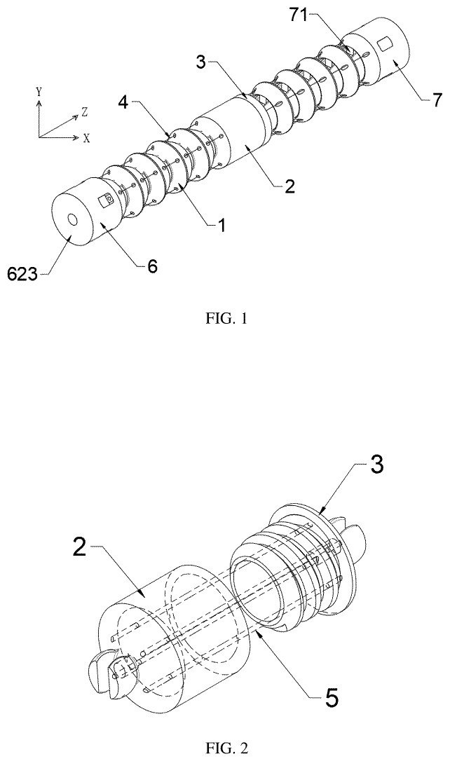

This embodiment provides a line-driven motion module, which may serve as a mechanical member for the purpose of medical treatment and diagnosis for use and certainly may be adapted to an application environment of a motion joint of a human body, as shown in FIG. 1 and FIG. 2, the line-driven motion module includes a joint structure, a positioning structure and two connecting lines 4, one end of the joint structure is configured to be connected with an external driving mechanism, the other end of the joint structure is configured to be connected with an external operating mechanism, and the joint structure includes a plurality of interconnected joint units 1; and the positioning structure is arranged between two adjacent joint units 1, and the connecting lines 4 are arranged in all the joint units 1 and the positioning structure in a penetrating manner and configured to adjust motion of the adjacent joint units 1 in response to motion of the external driving mechanism.

The positioning structure includes a first positioning member 2 and a second positioning member 3, the first positioning member 2 and the second positioning member 3 are movably connected to each other and can move towards or away from each other in a first direction, and the position structure actively adjusts a position of the joint structure connected thereto in the first direction; and each of the connecting lines 4 is arranged to pass through two ends of the positioning structure, and the first positioning member 2 and the second positioning member 3 are configured to limit and restrain a length of each of the connecting lines 4 inside the positioning structure.

In the line-driven motion module provided by this embodiment, when the first positioning member 2 and the second positioning member 3 move towards each other in the first direction, a length of each of the connecting lines 4 is limited and received in a line receiving cavity co-enclosed by the first positioning member 2 and the second positioning member 3; displacement of the connecting lines 4 in the first direction may be controlled through a relative motion position of the first positioning member 2 and the second positioning member 3 and is fitted with a position of the joint structure, an initial degree of tightness of each of the connecting lines 4 may be prevented from being interfered, withdrawal folding motion and extending and expanding motion of the joint structure are achieved through cooperation of the positioning structure and the connecting lines 4, a motion trajectory of the joint structure is optimized, a degree of freedom of motion of the line-driven motion module in the first direction and a degree of freedom of rotation thereof around the first direction are increased, thus a motion range of the joint structure is widened, and a purpose of enhancing the flexible operation performance of a minimally invasive device is achieved.

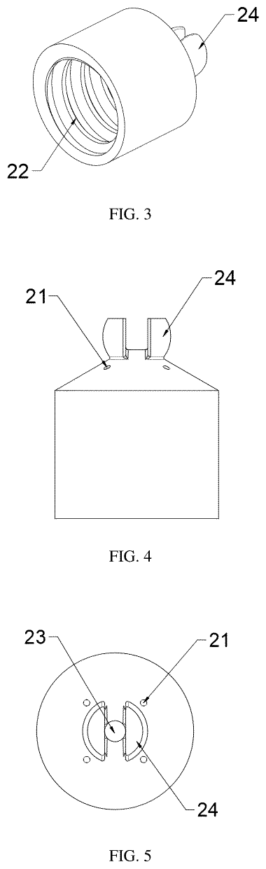

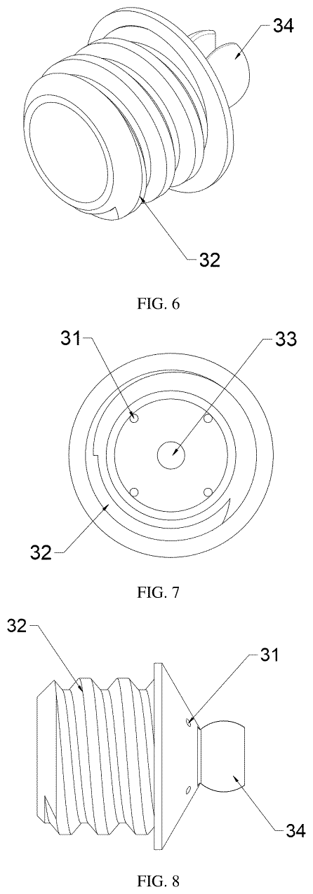

As shown in FIG. 3 to FIG. 8, the first positioning member 2 further includes first joining portions 24 arranged on a side, close to a proximal end, of the first positioning member 2, and the first joining portions 24 are movably connected to the adjacent joint units 1; the second positioning member 3 further includes second joining portions 34 arranged on a side, away from the proximal end, of the second positioning member 3, and the second joining portions 34 are movably connected to the adjacent joint units 1. The adjacent joint units 1 are driven to move through the first joining portions 24 and the second joining portions, so that the whole joint structure performs a withdrawal folding motion and an extending and expanding motion. The first joining portions 24 and the second joining portions may limit the adjacent joint unit 1, so it is guaranteed that the positioning structure and the adjacent joint unit 1 cooperate flexibly and stably, which is conducive to flexible bending of the joint structure.

As shown in FIG. 2, the positioning structure further includes positioning sleeves 5, the positioning sleeves 5 are configured to limit and receive the connecting lines 4, the number of the positioning sleeves 5 is consistent with the number of the connecting lines 4, the positioning sleeves 5 are arranged inside the line receiving cavity of the positioning structure, and two ends of each positioning sleeve 5 are connected with the corresponding connecting line 4 at a first line connecting port 21 of the first positioning member 2 and a second line connecting port 31 of the second positioning member 3, respectively. Motion of the connecting lines 4 are limited through cooperation of the first positioning member 2, the second positioning member 3 and the positioning sleeves 5, and the positioning sleeves 5 are configured to limit axial displacement of the connecting lines 4.

In this embodiment, the positioning sleeves 5 are flexible tubes. When the first positioning member 2 and the second positioning member 3 move towards each other in the first direction, the first positioning member 2 and the second positioning member 3 drive two ends of each positioning sleeve 5 to move towards each other relative to a middle of the positioning sleeve, and at the same time, the positioning sleeves 5 are configured to limit and squeeze the connecting lines 4 therein, so that the positioning sleeves 5 are coiled or curled synchronously with the connecting lines 4 inside the line receiving cavity; and when the first positioning member 2 and the second positioning member 3 move away from each other in the first direction, the first positioning member 2 and the second positioning member 3 drive the two ends of each positioning sleeve 5 to move away from each other relative to the middle of the positioning sleeve, at the same time, the two ends of the positioning sleeves 5 are configured to limit and stretch the connecting lines 4 therein, so that the connecting lines 4 are unfolded or stretched synchronously with the positioning sleeves 5, displacement of the connecting lines 4 matches a distance between the first positioning member 2 and the second positioning member 3, and the motion range of the joint structure can be widened. The line-driven motion module is in a bent state and an initial state, and a total length of each connecting line 4 limited inside the corresponding positioning sleeve 5 remains unchanged.

In this embodiment, as shown in FIG. 11, each joint unit 1 is provided with mounting holes 123 and is movably connected with a corresponding connecting line 4, and the connecting line 4 abuts against the mounting holes 123 so as to transmit the adjacent joint unit 1. The first line connecting port and the second line connecting port are mutually connected to the mounting holes in the close joint units through the connecting lines respectively.

In the line-driven motion module provided by the present application, as shown in FIG. 2 to FIG. 8, the first positioning member 2 is provided with a first connecting portion 22, and the second positioning member 3 is provided with a second connecting portion 32.

In this embodiment, the first connecting portion 22 and the second connecting portion are mated screw thread structures, when the first connecting portion 22 and the second connecting portion 32 are in threaded connection, the first positioning member 2 and the second positioning member 3 are relatively movable towards each other or away from each other in the first direction, the first direction being an axial direction (for example, a Z-axis direction shown in FIG. 1) of the screw thread structures.

In this embodiment, the first positioning member 2 and the second positioning member 3 are tubular structures which are nested together, the first positioning member 2 is rotationally nested on the second positioning member 3, and the first connecting portion 22 is formed on an inner wall surface of the first positioning member 2; the second connecting portion 32 is formed on an outer wall surface of the second positioning member 3; and the line receiving cavity is a cavity within the tubular structures.

When the motion range of the line-driven motion module is to be adjusted, during specific operation, the first positioning member 2 may be fixed, the second positioning member 3 may be rotated, so the line-driven motion module has a displacement in the first direction, and the joint structure adjacent to the second positioning member 3 rotates around the first direction; by fixing the second positioning member 3 and rotating the first positioning member 2, the line-driven motion module has a displacement in the first direction and the joint structure adjacent to the first positioning member 2 rotates around the first direction; by simultaneously rotating the first positioning member 2 and the second positioning member 3, the line-driven motion module has a displacement in the first direction and the joint structure adjacent to the first positioning member 2 and the second positioning member 3 respectively rotates around the first direction, and the three operation manners may be selected according to actual demands and working conditions.

In the line-driven motion module provided by the this embodiment, a rotation angle between the first positioning member 2 and the second positioning member 3 is relevant to the number of turns, and a relative rotation angle between the first positioning member 2 and the third positioning member 3 is not limited specifically here. In this embodiment, the number of turns for which the first positioning member 2 and the second positioning member 3 can rotate is set as three, the first positioning member 2 and the second positioning member 3 may be in threaded connection at any angle ranging from 0 degree to 1080 degrees, and the first positioning member 2 and the second positioning member 3 may be fixed through an external fixing connection member. Certainly, the first positioning member 2 may also be fixed to an external connector, a tooth member is externally arranged on the second positioning member 3 in a sleeving manner, an external driving member drives the tooth member so that the tooth member drives the second positioning member 3 to rotate relative to the first positioning member 2, and thus the relative rotation angle between the first positioning member 2 and the second positioning member 3 changes in a range from 0 degree to 1080 degrees. Correspondingly, the second positioning member 3 may be fixed to the external connector, a gear member is externally arranged on the first positioning member in a sleeving manner, the external driving member drives the gear member so that the gear member drives the first positioning member 2 to rotate relative to the second positioning member 3, and thus the relative rotation angle between the first positioning member 2 and the second positioning member 3 changes in a range from 0 degree to 1080 degrees.

In the line-driven motion module provided by this embodiment, the screw thread structures adopt constant pitch screw threads, a length of extending or withdrawing the connecting lines 4 is equal to the relative displacement between the first positioning member 2 and the second positioning member 3, the number of rotation turns is in a direct-proportion linear relation with a screw pitch of the screw thread structure as well as a length variable quantity of the connecting line 4. The screw pitch is configured according to a length variable quantity of the connecting line 4 needed in working conditions.

A whole length and a material of the positioning structure are not limited specifically, a material of the first positioning member 2 and the second positioning member 3 may be steel, medical plastics and the like, and a length and a material thereof may be selected according to actual application occasions.

The line-driven motion module provided by this embodiment, as shown in FIG. 1, further includes a first adapter structure 6 and a second adapter structure 7 which are spaced apart from each other, the first adapter structure 6 and the second adapter structure 7 are arranged at two ends of the joint structure, the first adapter structure 6 is configured to be connected with the external driving mechanism, and the second adapter structure 7 is configured to be connected with the operating mechanism; and the positioning structure is mounted between the first adapter structure 6 and the second adapter structure 7.

In this embodiment, the line-driven motion module has bent states in which the first adapter structure 6 draws the connection lines 4 under an external force to enable the second adapter structure 7 and the joint structure to bend in a linked manner synchronously with respect to the first adapter structure 6.

In this embodiment, the line-driven motion module has an initial state in which the first adapter structure 6 draws the connecting lines 4 under an external force so as to drive the first adapter structure 6, the positioning structure and the second adapter structure 7 to be arranged coaxially.

The external driving mechanism drives and adjusts the motion of the first adapter structure 6, the second adapter structure 7 is transmitted through cooperation of the connecting lines 4 and the joint structure, and the line-driven motion module may be switched between the bent state and the initial state. In the bent state and the initial state, the first adapter structure 6 and the second adapter structure 7 move correspondingly around the positioning structure. In the initial state, all the connecting lines 4 may be arranged in the same length, which is conducive to synchronous linkage of the second adapter structure 7 and the first adapter structure 6, and then the second adapter structure 7 achieves the expected motion actions and positions. Through the integral module design, stability of the connecting lines 4 driving the joint units 1 is improved, a connection strength of the joint structure is better improved, and whole motion performance of the line-driven motion module is balanced.

In the line-driven motion module provided by this embodiment, as shown in FIG. 1, two or more connecting lines 4 are arranged, and all the connecting lines 4 are arranged in rotation symmetry in an axial direction of the positioning structure. The connecting lines 4 are configured to drive the joint structure to rotate, so that the adjacent joint unit 1 cooperates flexibly. In this embodiment, the connecting lines 4 may be selected as an elastic fiber rope, a nickel-titanium wire and other elastic materials, so that the connecting lines 4 drive a joint structure and the second adapter structure 7, and thus a linkage function of the line-driven motion module is achieved.

In this embodiment, as shown in FIG. 1, taking four connecting lines 4 as an example, the four connecting lines 4 are distributed on the line-driven motion module in a penetrating manner. In this embodiment, the two ends of each connecting line 4 are fixedly connected with the first adapter structure 6 and the second adapter structure 7 respectively, and the connecting lines 4 are arranged on all the joint units 1 and the positioning structure in a penetrating manner and are movably connected with the joint units 1 and the positioning structure.

In the line-driven motion module provided by this embodiment, in the initial state, all the connecting lines 4 have the same length, so that the connecting lines 4 are drawn by motion of the first adapter structure 6 to correspondingly adjust the adjacent joint unit 1 and the second adapter structure 7, so as to be fitted with motion of the first adapter structure 6, the line-driven motion module abuts against the positioning structure through the connecting lines 4, and when the first adapter structure 6 draws one end of each connecting line 4 to move, the second adapter structure 7 where the other end of the corresponding connecting line 4 is located is enabled to perform fitting actions, and thus a linkage bending process is achieved. The connecting lines 4 are tightly and fixedly connected between the first adapter structure 6 and the second adapter structure 7, and the whole line-driven motion module has a good joint strength.

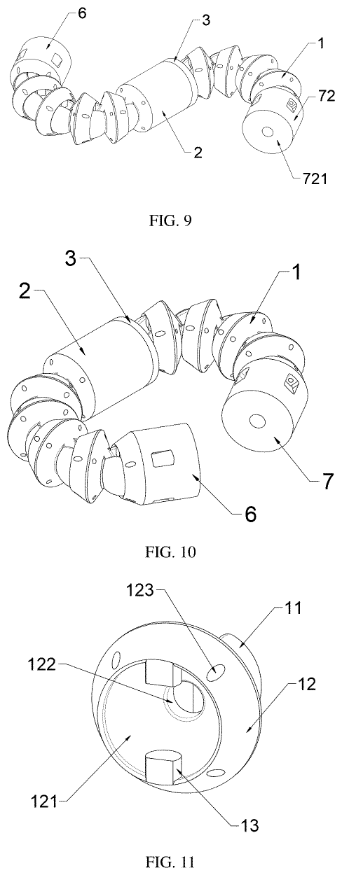

As shown in FIG. 13, the first adapter structure 6 includes a first adapter body 62. As shown in FIG. 1, the second adapter structure 7 includes a second adapter body 72, and the first adapter body 62 and the second adapter body 72 are spaced apart from each other; and the first adapter body 62 has a first end surface 623 remote from a side thereof connected to the joint unit 1, the second adapter body 72 has a second end surface 723 remote from a side thereof connected to the joint unit 1. As shown in FIG. 1, the first end surface 623 is located on a proximal end portion of the line-driven motion module. As shown in FIG. 9, the second end surface 721 is located on a distal end portion of the line-driven motion module, and in the initial state of the line-driven motion module, the first end surface 623 is arranged opposite to the second end surface 721.

It needs to be noted that when the line-driven motion module is driven to move specifically, the first end surface 623 may be used as a reference surface of driving of the driving mechanism, the driving mechanism drives the first adapter structure 6 for position adjustment, the whole line-driven motion module moves in a three-dimensional space to implement corresponding bending action posture, then the first end surface 623 is adjusted through the driving mechanism so that the first end surface 623 and the second end surface 721 perform corresponding actions, a purpose of adjusting the position and orientation of the second end surface 721 is achieved, and thus the operating mechanism on the second adapter structure 7 can perform actions of a pre-determined target position and target direction. Correspondingly, the first end surface 623 is adjusted through the driving mechanism so that the second end surface 721 performs actions corresponding to the first end surface 623, then the driving mechanism drives the first adapter structure 6 for position adjustment, and thus the whole line-driven motion module moves in the three-dimensional space to implement the corresponding bending action posture. The first end surface 623 may be used as a mounting surface for being connected with the driving mechanism, and a shape of the first end surface 623 may be a plane, a curved surface or a stepped surface, which is not limited specifically here.

In this embodiment, the first adapter structure 6 and the second adapter structure 7 are the same, taking the first adapter structure 6 as an example, as shown in FIG. 13 and FIG. 14, the first adapter structure 6 further includes fitting holes 622, the connecting lines 4 are arranged and fixed in the fitting holes 622 in a penetrating manner, and a connection between the fitting holes 622 and the connecting lines 4 includes but is not limited to a welding connection, a bonding connection or locking through a fastening connector.

As shown in FIG. 1, FIG. 13 and FIG. 14, the first adapter structure 6 includes a third joining portion 61, and the third joining portion is movably connected with a joint unit 1. The second adapter structure 7 includes a fourth joining portion 71, and the fourth joining portion 71 is movably connected with a joint unit 1. The third joining portion 61 cooperates with the connecting lines 4 so as to transmit motive power and torque of the first adapter structure 6 to the joint structure; and the fourth joining portion 71 cooperates with the connecting lines 4 so as to receive the motive power and the torque and transmit the motive power and the torque to the second adapter structure 7, and thus the second adapter structure 7 changes in a linked manner according to the motion position of the first adapter structure 6.

In this embodiment, the number of joint units between the positioning structure and the first adapter structure 6 and the number of joint units between the positioning structure and the second adapter structure 7 are not limited specifically. FIG. 1 shows a use situation in this embodiment where the number of joint units 1 at one end of the positioning structure is the same as the number of joint units at the other end of the positioning structure.

A joint structure between the first adapter structure 6 and the positioning structure is named a first joint structure, and a joint structure between the second adapter structure 7 and the positioning structure is named a second joint structure. It needs to be noted that in the line-driven motion module, the first adapter structure 6 and the first joint structure may bend relative to the second adapter structure 7 and the second joint structure, a displacement generated by the driving mechanism acting on the first adapter structure 6 in a three-dimensional space relative to the positioning structure is implemented by compensation through mutual rotation of the adjacent interconnected joint units 1 between the first adapter structure 6 and the positioning structure and self-extending and withdrawing of the connecting lines 4, so that the first joint structure performs flexible bending and stretching actions; and in this case, the first end surface 623 and the second end surface 721 remain an original position relationship in parallel in space, and the second adapter structure 7 and the second joint structure remain an original pose without a change. In a flexible motion action of the first joint structure, the driving mechanism may drive the first adapter structure 6, so that the line-driven motion module performs bending and stretching adjustment on the first joint structure in a position of a bending action process of the line-driven motion module, thus the first joint structure may achieve the expected motion pose and the position during the bending and stretching action process, so as to meet the demanded working condition. Correspondingly, the driving mechanism may be caused to drive the first adapter structure 6 so as to perform bending and stretching adjustment on the first joint structure, then the driving mechanism is caused to drive the first adapter structure 6 so as to cause the whole line-driven motion module to generate a bending action, and thus the demanded working condition is met.

It needs to be noted that the line-driven motion module may use any of actions poses that the line-driven motion module can achieve as an initial position of driving of the driving mechanism, and the line-driven motion module has advantages of flexible operation and consecutive and stable motion.

In the line-driven motion module provided by this embodiment, each connecting line 4 is arranged in a corresponding first line connecting port 21 of the first positioning member 2 and a corresponding second line connecting port 31 of the second positioning member 3 in a penetrating manner; and when the first positioning member 2 is screwed to a first rotation position relative to the second positioning member 3, each first line connecting port 21 and the corresponding second line connecting port 31 are on a line in the same direction as the first direction. As shown in FIG. 9, the line-driven motion module has a first bent state in which the first adapter structure 6 draws the connection lines 4 under an external force to enable the second adapter structure 7 and the joint structure to bend in a linked manner to move in an S shape synchronously with respect to the first adapter structure 6. The external driving mechanism drives the first adapter structure 6, so that the first adapter structure 6 and the second adapter structure 7 move towards each other in opposite directions in an X-Y plane, and the first adapter structure 6 and the second adapter structure 7 relatively bend reversely. As shown in FIG. 9, the first end surface 623 and the second end surface 721 are arranged in parallel, and by operating the first end surface 623, the second end surface 721 performs the corresponding actions synchronously.

When the first positioning member 2 rotates relative to the second positioning member 3 and the first positioning member 2 is screwed to a second rotation position relative to the second positioning member 3, a first plane containing one first line connecting port 21 and the first direction is oriented at an angle of 90 degrees to a second plane containing the corresponding second line connecting port 31 and the first direction. As shown in FIG. 10, the line-driven motion module is in a second bent state in which the first adapter structure 6 draws the connection lines 4 under an external force to enable the second adapter structure 7 and the joint structure to bend in a linked manner to move in a noncoplanar S shape synchronously with respect to the first adapter structure 6. When external driving mechanism drives the first adapter structure 6 to move in an X-axis extending direction shown in FIG. 1, the first adapter structure 6 and the first joint structure perform the bending motion jointly in the X-axis extending direction, in this case, the second adapter structure 7 and the second joint structure perform the bending motion jointly in a Y-axis extending direction shown in FIG. 1 under a transmission action of the connecting lines 4, a projection of a bending direction of the first joint structure in the X-Y plane is oriented at an angle of 90 degrees to a projection of the bending direction of the second joint structure in the X-Y plane, so that the joint assemblies may achieve the expected action poses and positions of a noncoplanar S-shaped curved action process, so as to meet the demanded working conditions.

When the first positioning member 2 rotates relative to the second positioning member 3 and the first positioning member 2 is screwed to a third rotation position relative to the second positioning member 3, a first plane containing one first line connecting port 21 and the first direction is oriented at an angle of 180 degrees to a second plane containing the corresponding second line connecting port 31 and the first direction. The line-driven motion module is in a third bent state in which the first adapter structure 6 draws the connection lines 4 under an external force to enable the second adapter structure 7 and the joint structure to bend in a linked manner to move in a C shape synchronously with respect to the first adapter structure 6. The external driving mechanism drives the first adapter structure 6 so that the first adapter structure 6 and the second adapter structure 7 move towards each other in a circumferential plane along a radial plane of a positioning body, so that the joint assemblies may achieve the expected actions and positions, so as to meet the expected working conditions.

The line-driven motion module provided by this embodiment further includes a swiveling state in which the first adapter structure 6 is rotated under an external force to draw the connecting lines 4 and enable the second adapter structure 7 and the joint structure to twist synchronously with the first adapter structure 6. In this embodiment, during whole swiveling rotation, the first positioning member 2 and the second positioning member 3 are fixed, the positioning structure is rotationally connected to an externally-connected supporting ring, so as to achieve the purpose that the line-driven motion module rotates around an axis of the positioning structure.

Taking an implementation as an example, a rotation limit position of the first positioning member 2 and the second positioning member 3 is the third rotation position, thus, during a rotation process of the first positioning member 2 and the second positioning member 3, the first line connecting port 21 and the second line connecting port 31 may pass through corresponding positions of the first rotation position and the second rotation position many times, so various rotation positions of the first positioning member 2 and the second positioning member 3 may be met by using one positioning structure, such as forms of the first rotation position, the second rotation position and the third rotation position, and thus demands of a user are met.

In other variant implementations, the first rotation position may also be used as the rotation limit position of the first positioning member 2 and the second positioning member 3, and correspondingly, the second rotation position and the third rotation position may be process positions. Likewise, the second rotation position may be the rotation limit position of the first positioning member 2 and the second positioning member 3, and correspondingly, the first rotation position and the third rotation position may be process positions.

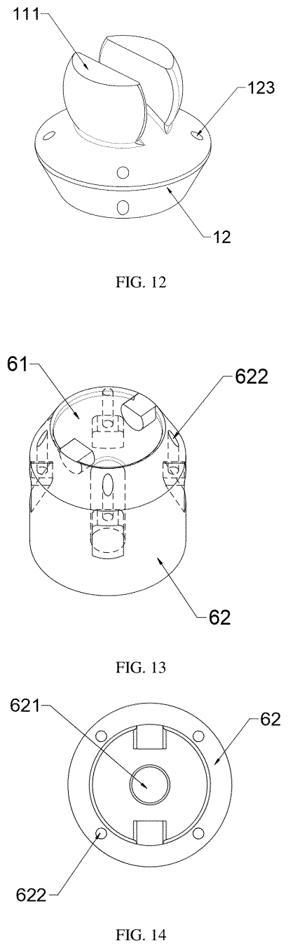

In the line-driven motion module provided by this embodiment, as shown in FIG. 11 and FIG. 12, each joint unit 1 includes a rotating member 11 and a limiting base 12. The rotating member 11 is provided with limiting portions 111 in a circumferential direction. The limiting base 12 is fixedly connected to the rotating member 11. The limiting base 12 is internally provided with a receiving cavity 121 for being movably connected to the rotating member 11 of an adjacent joint unit 1. The receiving cavity 121 is further internally provided with limiting members 13, and the limiting members 13 cooperate with the limiting portions 111 of the adjacent joint unit 1 to limit axial rotation of the rotating member 11 of the adjacent joint unit 1. The adjacent joint unit 1 has two perpendicular pivots, one pivot is parallel to an extending direction of one limiting member 13, and the other pivot is parallel to an intersection line between a radial plane of the limiting member 13 and the extending direction of the limiting member 13.

In this embodiment, the rotating member 11 is a sphere, an inner wall surface of the receiving cavity 121 is fitted with an outer wall surface of the rotating member 11 of the adjacent joint unit 1. Two limiting portions 111 are provided, and the two limiting portions 111 cooperate to form the sphere. The two limiting portions 111 are spaced apart to form a fitting space for the limiting members 13, and the limiting members 13 are movably connected into the fitting space.

In the line-driven motion module provided by this embodiment, as shown in FIG. 3 and FIG. 5, the first positioning member 2 includes a first connecting hole 23 extending in the same direction as an extending direction of the first positioning member 2. As shown in FIG. 7, the second positioning member 3 includes a second connecting hole 33 extending in the same direction as an extending direction of the second positioning member 3. As shown in FIG. 13 and FIG. 14, each of the adapter structures is provided with a third connecting hole 621 extending in the same direction as an extending direction of the adapter structure. As shown in FIG. 11 and FIG. 12, each of the joint units 1 is provided with a core hole 122, and all the core holes 122, all the second connecting holes 33 and all the first connecting holes 23 together form an intervention channel configured for an externally-connected pipe to penetrate therethrough. Fitting holes are configured in the adapter structures, the joint structure and the positioning structure, which is intended to form an intervention channel for the purpose of medical treatment, a flexible pipe may be connected into the intervention channel through an operation space formed by the intervention channel, and when the line-driven motion module has a bending change, the pipe also bends, so as to meet the expected working conditions.

In the line-driven motion module provided by this embodiment, the first joining portion 24 and the second joining portion 34 are respectively identical in structure to the rotating member 11, and the third joining portion 61 and the fourth joining portion 71 are respectively identical in structure to the limiting base 12. The joining portions play a role in transferring motive power and torque, so as to transmit linkage of the connecting lines 4 for the joint structure and the adapter structures. Meanwhile, the joining structures are used for limited rotation connection of the joint structure.

In an alternative implementation by this embodiment, the first joining portion 24 and the second joining portion 34 are respectively identical in structure to the limiting base 12, and the third joining portion 61 and the fourth joining portion 71 are identical in structure to the rotating member 11.

As an alternative implementation in this embodiment, the third positioning member 2 is provided with a third connecting portion, and the second positioning member 3 is provided with a fourth connecting portion. The third connecting portion and the fourth connecting portion are mated slidable structures. When the third connecting portion and the fourth connecting portion slide relative to each other, the first positioning member 2 and the second positioning member 3 relatively move towards each other or away from each other in the first direction, and the first direction is a sliding direction of the slidable structures. The third connecting portion and the fourth connecting portion slide in cooperation, so the first positioning member 2 and the second positioning member 3 move towards each other or away from each other, so that a joint structure connected to the first positioning member 2 and a joint structure connected to the second positioning member 3 perform withdrawal folding motion or stretching and unfolding motion, the positioning sleeves 5 may perform bending motion in the receiving cavity co-enclosed by the first positioning member 2 and the second positioning member 3. When the first positioning member 2 and the second positioning member 3 relative move towards each other in the first direction, the positioning sleeves 5 and the connecting lines 4 bend and fold jointly. When the first positioning member 2 and the second positioning member 3 relatively move away from each other in the first direction, the positioning sleeves 5 and the connecting lines 4 are stretched and unfolded jointly.

As an alternative implementation of this embodiment, the first connecting portion 22 and the second connecting portion 32 are mated screw thread structures. The screw thread structures have variable pitch screw threads. The connecting lines 4 are directly arranged inside the first line connecting port 21 and the second line connecting port 31 in a penetrating manner. When the first positioning member 2 and the second positioning member 3 move towards each other in the first direction, the screw thread structures are in screwing-in action, a Relative elongation of each of the connecting lines 4 is limited and received in the line receiving cavity along the screw thread structures with the variable pitch screw threads, and the joint structures at the two ends of the positioning structure are driven by the first positioning member 2 and the second positioning member 3 to move towards each together so as to perform withdrawal folding. When the first positioning member 2 and the second positioning member 3 move away from each other in the first direction, the screw thread structures are in screwing-out action, the connecting lines 4 are limited and received in the line receiving cavity, and the joint structures at the two ends of the positioning structure are driven by the first positioning member 2 and the second positioning member 3 to move away from each other so as to perform stretching and unfolding. A variable quantity of a length of each connecting line 4 is the same as a screw pitch of the variable pitch screw threads, the screw thread structures limit the connecting lines, and thus influence of the lengths of the connecting lines 4 on the line-driven motion module is eliminated by using the variable pitch screw threads. It needs to be noted that in this case, a maximum angle of relative rotation of the first connecting portion 22 and the second connecting portion 32 is 180 degrees.

In an alternative implementation in this embodiment, in the positioning structure, the first joining portion 24 and the second joining portion 34 may be formed by the joint units 1, so as to achieve the purpose of transferring the motive power and the torque. One or two joint units 1 are directly fixed at the ends of the positioning structure in the first direction, a fixed manner may be integrated forming, welding, threaded connection, plug-pin connection or the like, and the joint units 1 and the joint structure are movably connected in series.

In an alternative implementation in this embodiment, in the third joining portion 61 of the first adapter structure 6 and the fourth joining portion 71 of the second adapter structure 7, each joining portion may be formed by the joint unit 1, so as to achieve the purpose of transferring the motive power and torque. One joint unit 1 is directly fixed at a distal end of the first adapter structure 6 or a proximal end of the second adapter structure 7, a fixed manner may be integrated forming, welding, threaded connection, plug-pin connection or the like, and the joint unit 1 and the joint structure are movably connected in series.

In the line-driven motion module provided in this embodiment, taking the first positioning member 2 and the second positioning member 3 moving towards to adjust a motion position of the joint structure as an example, a use process is as follows: the first positioning member 2 is fixed to the outside, the first positioning member 2 and the second positioning member 3 move towards each other by rotating the second positioning member 3, two ends of each positioning sleeve 5 are driven by the first positioning member 2 and the second positioning member 3 to move towards a middle segment of the positioning sleeve 5, at the same time, the positioning sleeve 5 generates curling motion to limit and squeeze the connecting lines 4, so the connecting lines 4 also generate curling motion, the joint structure connected to the first positioning member 2 is in an original position and pose, the joint structure connected to the second positioning member 3 rotates along with rotation of the second positioning member 3 and moves towards the first positioning member 2, in this case, the whole joint structure is in a shortened folding pose relative to the original joint structure, and the whole line-driven motion module generates withdrawal motion in the first direction.

In the line-driven motion module provided by this embodiment, the first positioning member 2 and the second positioning member 3 are movably connected to each other to move towards or away from each other in a first direction, the joint structure connected to the first positioning member 2 and the joint structure connected to the second positioning member 3 perform corresponding displacements of moving towards or away from each other in the first direction, and the motion position of the joint structure is adjusted by motion of the first positioning member 2 and the second positioning member 3, so as to achieve the purpose of adjusting the motion trajectory of the joint structure in the direction through the positioning structure. A length of each of the connecting lines 4 connected into the positioning structure is limited to be constant through positioning cooperation of the first positioning member 2 and the second positioning member 3, and specifically, when the first positioning member 2 and the second positioning member 3 move towards each other in the first direction, a length of each of the connecting lines (4) is limited in the line receiving cavity, and the joint structure performs withdrawal folding motion. When the first positioning member 2 and the second positioning member 3 move away from each other in the first direction, the length of each of the connecting lines 4 is released out of the line receiving cavity, in this case, the connecting lines 4 are extended and straightened, and the joint structure performs stretching and unfolding motion. When the first positioning member 2 and the second positioning member 3 limit a length of each of the connecting lines 4 in a distance between two ends inside the positioning structure to be constant, the length of each of the connecting lines 4 inside the positioning structure is constant all the time and does not change even through the first positioning member 2 and the second positioning member 3 slide in the first direction, and change motion of the connecting lines 4 inside the positioning structure is adapted to relative motion of the first positioning member 2 and the second positioning member 3 and the positions of the motion trajectory of the joint structure at the two ends of the positioning structure, which optimizes the motion trajectory of the joint structure, widens the motion range of the joint structure and achieves the purpose of enhancing the flexible operation performance of the minimally invasive device.

### Embodiment 2

This embodiment provides a pair of minimally invasive surgical forceps, including the line-driven motion module in Embodiment 1, thereby having the advantages brought by the line-driven motion module. The line-driven motion module may have a function of causing the joint structure to move in a first direction and a function of driving joint structures on a first positioning member 2 and a second positioning member 3 to rotate through rotation of a positioning structure, a motion range of the joint structure is widened effectively, and thus the minimally invasive surgical forceps can have a function of corresponding motion and rotation, and flexible operation of the minimally invasive surgical forceps is facilitated. In addition, an integrated module structure makes whole connection compact and connection strength high and can facilitate a fitting process of the minimally invasive surgical forceps and enhance stability during a use process.

Apparently, the above embodiments are merely examples for clear description, but not for limiting the implementations. Those ordinarily skilled in the art can also make modifications or variations in other different forms based on the above description. All implementations do not need to be and cannot be exhaustively cited here. Apparent modifications or variations derived from this still fall within the protection scope of the present application.

1. A line-driven motion module, comprising:
a joint structure, wherein one end of the joint structure is configured to be connected with an external driving mechanism, the other end of the joint structure is configured to be connected with an external operating mechanism, and the joint structure comprises a plurality of interconnected joint units;
a positioning structure, wherein the positioning structure is arranged between two adjacent joint units, the positioning structure comprises a first positioning member and a second positioning member, and the first positioning member and the second positioning member are movably connected to each other and can move towards or away from each other in a first direction; and
at least two connecting lines, wherein the connecting lines are arranged in all the joint units and the positioning structure in a penetrating manner, each of the connecting lines is arranged to pass through two ends of the positioning structure, and the first positioning member and the second positioning member are configured to limit and restrain a length of each of the connecting lines inside the positioning structure;
wherein, when the first positioning member and the second positioning member move towards each other in the first direction, a length of each of the connecting lines is limited and received in a line receiving cavity co-enclosed by the first positioning member and the second positioning member; and when the first positioning member and the second positioning member move away from each other in the first direction, the length of each of the connecting lines is straightened in the line receiving cavity.

2. The line-driven motion module according to claim 1, wherein:
the positioning structure further comprises at least two positioning sleeves,
the positioning sleeves are arranged in correspondence with the connecting lines and configured to limit and receive the connecting lines,
the positioning sleeves are arranged inside the line receiving cavity, and
two ends of each positioning sleeve are connected with a corresponding connecting line at a first line connecting port of the first positioning member and at a second line connecting port of the second positioning member, respectively.

3. The line-driven motion module according to claim 2, wherein:
the positioning sleeves are flexible tubes;
when the first positioning member and the second positioning member move towards each other in the first direction, the flexible tubes are folded or coiled or curled synchronously with the connecting lines inside the line receiving cavity; and
when the first positioning member and the second positioning member move away from each other in the first direction, the flexible tubes are unfolded or stretched synchronously with the connecting lines.

4. The line-driven motion module according to claim 1, wherein:
the line-driven motion module further comprises a first adapter structure and a second adapter structure which are spaced apart from each other, the first adapter structure and the second adapter structure are arranged at two ends of the joint structure, the first adapter structure is configured to be connected with the external driving mechanism, and the second adapter structure is configured to be connected with the operating mechanism; and
the line-driven motion module has bent states in which the first adapter structure draws the connection lines under an external force to enable the second adapter structure and the joint structure to bend in a linked manner synchronously with respect to the first adapter structure, and an initial state in which the first adapter structure, the positioning structure and the second adapter structure are arranged coaxially; and the line-driven motion module is arranged to switch between the bent states and the initial state.

5. The line-driven motion module according to claim 4, wherein:
the first positioning member is provided with a first connecting portion, and the second positioning member is provided with a second connecting portion; and
the first connecting portion and the second connecting portion are mated screw thread structures, and when the first connecting portion and the second connecting portion are in threaded connection, the first positioning member and the second positioning member are relatively movable towards each other or away from each other in the first direction, the first direction being an axial direction of the screw thread structures.

6. The line-driven motion module according to claim 5, wherein:
the first positioning member and the second positioning member are tubular structures which are nested together;
the first connecting portion is formed on an inner wall surface of the first positioning member;
the second connecting portion is formed on an outer wall surface of the second positioning member; and
the line receiving cavity is a cavity within the tubular structures.

7. The line-driven motion module according to claim 4, wherein:
each connecting line is arranged in a corresponding first line connecting port of the first positioning member and a corresponding second line connecting port of the second positioning member in a penetrating manner; and
when the first positioning member is screwed to a first rotation position relative to the second positioning member, each first line connecting port and the corresponding second line connecting port are on a line in the same direction as the first direction, and the line-driven motion module is in a first bent state in which the first adapter structure draws the connection lines under an external force to enable the second adapter structure and the joint structure to bend in a linked manner to move in an S shape synchronously with respect to the first adapter structure.

8. The line-driven motion module according to claim 7, wherein, in the first bent state, a first end surface, remote from a side thereof connected to the joint structure, of the first adapter structure, is arranged parallel to a second end surface, remote from a side thereof connected to the joint structure, of the second adapter structure.

9. The line-driven motion module according to claim 5, wherein:
each connecting line is arranged in a corresponding first line connecting port of the first positioning member and a corresponding second line connecting port of the second positioning member in a penetrating manner; and
when the first positioning member is screwed to a second rotation position relative to the second positioning member, a first plane containing one first line connecting port and the first direction is oriented at an angle of 90 degrees to a second plane containing the corresponding second line connecting port and the first direction, and the line-driven motion module is in a second bent state in which the first adapter structure draws the connection lines under an external force to enable the second adapter structure and the joint structure to bend in a linked manner to move in a noncoplanar S shape synchronously with respect to the first adapter structure.

10. The line-driven motion module according to claim 5, wherein:
each connecting line is arranged in a corresponding first line connecting port of the first positioning member and a corresponding second line connecting port of the second positioning member in a penetrating manner; and
when the first positioning member is screwed to a third rotation position relative to the second positioning member, a first plane containing one first line connecting port and the first direction is oriented at an angle of 180 degrees to a second plane containing the corresponding second line connecting port and the first direction, and the line-driven motion module is in a third bent state in which the first adapter structure draws the connection lines under an external force to enable the second adapter structure and the joint structure to bend in a linked manner to move in a C shape synchronously with respect to the first adapter structure.

11. The line-driven motion module according to claim 4, wherein:
the first positioning member is provided with a third connecting portion, and the second positioning member is provided with a fourth connecting portion; and
the third connecting portion and the fourth connecting portion are mated slidable structures, when the third connecting portion and the fourth connecting portion slide relative to each other, the first positioning member and the second positioning member relatively move towards each other or away from each other in the first direction, and the first direction is a sliding direction of the slidable structures.

12. The line-driven motion module according to claim 8, wherein, the line-driven motion module further comprises a swiveling state in which the first adapter structure is rotated under an external force to draw the connecting lines and enable the second adapter structure and the joint structure to twist synchronously with the first adapter structure.

13. The line-driven motion module according to claim 12, wherein each joint unit comprises:
a rotating member provided with limiting portions in a circumferential direction; and
a limiting base fixedly connected to the rotating member, wherein the limiting base is internally provided with at least one receiving cavity for being movably connected to the rotating member of an adjacent joint unit, the receiving cavity is further internally provided with limiting members, and the limiting members cooperate with the limiting portions of the adjacent joint unit to limit axial rotation of the rotating member of the adjacent joint unit.

14. The line-driven motion module according to claim 13, wherein:
the rotating member is a sphere, an inner wall surface of the receiving cavity is fitted with an outer wall surface of the rotating member of the adjacent joint unit;
two limiting portions are provided, and the two limiting portions cooperate to form the sphere; and
the two limiting portions are spaced apart to form a fitting space for limiting the limiting members, and the limiting members are movably connected into the fitting space.

15. The line-driven motion module according to claim 13, wherein:
the first positioning member comprises a first connecting hole extending in the same direction as an extending direction of the first positioning member;
the second positioning member comprises a second connecting hole extending in the same direction as an extending direction of the second positioning member;
each adapter structure is provided with a third connecting hole extending in the same direction as an extending direction of the adapter structure; and
each of the joint units is provided with a core hole, and all the core holes, all the second connecting holes and all the first connecting holes together form an intervention channel configured for an externally-connected pipe to penetrate therethrough.

16. The line-driven motion module according to claim 15, wherein:
the first positioning member further comprises a first joining portion arranged on a side, close to a proximal end, of the first positioning member, and the first joining portion is movably connected to the joint structure;
the second positioning member further comprises a second joining portion arranged on a side, away from the proximal end, of the second positioning member, and the second joining portion is movably connected to the joint structure; and
the first joining portion and the second joining portion are respectively identical in structure to the rotating member or identical in structure to the limiting base.

17. The line-driven motion module according to claim 16, wherein:
the first adapter structure comprises a third joining portion movably connected with a joint unit;
the second adapter structure comprises a fourth joining portion movably connected with a joint unit; and
the third joining portion and the fourth joining portion are respectively identical in structure to the rotating member or identical in structure to the limiting base.

18. Minimally invasive surgical forceps, comprising the line-driven motion module of claim 1.
